(19) United States
(12) Patent Application Publication
(10) Pub. No.: US 2009/0186059 A1
(43) Pub. Date: Jul. 23, 2009

(54) DEVICES AND METHODS FOR ELUTION OF NUCLEIC ACID DELIVERY COMPLEXES

(76) Inventors: Elizabeth E. Johnson, Minneapolis, MN (US); Aron Brent Anderson, Minnetonka, MN (US); Joseph Schmidt McGonigle, Minneapolis, MN (US)

Correspondence Address: Pauly, Devries Smith & Deffner, L.L.C., Plaza VII, 45 South Seventh Street, Suite 3000, Minneapolis, MN, 55402-1630, US

(21) Appl. No.: 12/353,792

(22) Filed: Jan. 14, 2009

Related U.S. Application Data
(60) Provisional application No. 61/020,856, filed on Jan. 14, 2008.

(51) Int. Cl. A61K 31/7088; A61K 9/00

(52) U.S. Cl. 424/422; 514/44

(57) ABSTRACT

Embodiments of the invention include devices and methods for the controlled elution of nucleic acid delivery complexes. In an embodiment, the invention includes a method of making a medical device. The method can include complexing nucleic acids with a carrier agent to form a delivery complex solution, applying the delivery complex solution to a substrate, and applying a polymeric solution to the substrate. In another embodiment, the invention includes a method of making a medical device including complexing nucleic acids with a carrier agent to form nucleic acid delivery complexes, combining the nucleic acid delivery complexes with a polymer solution and a cross-linking agent, wherein the cross-linking agent is positively charged or charge neutral. In an embodiment, the invention includes an implantable medical device including a substrate and a coating disposed on a surface of the substrate, the coating comprising a polymeric matrix and a plurality of disperse nucleic acid delivery complexes disposed within the polymeric matrix. Other embodiments are included herein.

This application claims the benefit of U.S. Provisional Application No. 61/020,856, filed Jan. 14, 2008, the content of which is herein incorporated by reference.

### FIELD OF THE INVENTION

The present invention relates to devices and methods for the controlled elution of active agents. More specifically, the present invention relates to devices and methods for the controlled elution of nucleic acid delivery complexes.

### BACKGROUND OF THE INVENTION

One promising approach to the treatment of various medical conditions is the administration of nucleic acids as therapeutic agents. By way of example, this approach can include the administration of RNA, DNA, siRNA, miRNA, piRNA, shRNA, antisense nucleic acids, aptamers, ribozymes, catalytic DNA and the like.

Unfortunately, nucleic acid therapy has proven to be difficult to implement. In order to mediate an effect on a target cell, a nucleic acid based active agent must generally be delivered to an appropriate target cell, taken up by the cell, released from an endosome, and transported to the nucleus or cytoplasm (intracellular trafficking), among other steps. As such, successful treatment with nucleic acids depends upon site-specific delivery, stability during the delivery phase, and a substantial degree of biological activity within target cells. In addition, successful therapy with nucleic acids using non-viral delivery systems can also depend on maintaining a therapeutically effective dose of the nucleic acids in a target tissue over an extended period of time. However, the steps of delivery, uptake, and maintenance of an effective dose over time can all be complicated by the fact that nucleic acids are readily degraded by enzymes in the in vivo environment, amongst other issues.

Accordingly, a need remains for devices and methods that can deliver therapeutic nucleic acids to a target tissue over an extended period of time in a site specific manner, all while preserving activity of the nucleic acids.

### SUMMARY OF THE INVENTION

Embodiments of the invention include devices and methods for the controlled elution of nucleic acid delivery complexes. In an embodiment, the invention includes a method of making a medical device. The method can include complexing nucleic acids with a carrier agent to form a delivery complex solution comprising nucleic acid delivery complexes, applying the delivery complex solution to a substrate, and applying a polymeric solution to the substrate.

In an embodiment, the invention includes a method of making a medical device. The method can include complexing nucleic acids with a carrier agent to form nucleic acid delivery complexes, combining the nucleic acid delivery complexes with a polymer solution and a cross-linking agent, wherein the cross-linking agent is positively charged or charge neutral.

In an embodiment, the invention includes an implantable medical device including a substrate and a coating disposed on a surface of the substrate, the coating comprising a polymeric matrix and a plurality of disperse nucleic acid delivery complexes disposed within the polymeric matrix. The polymeric matrix can include a degradable polymer and a non-degradable polymer. The nucleic acid delivery complexes can include a nucleic acid and a carrier agent complexed to the nucleic acid. The coating can be configured to elute the nucleic acid delivery complexes in vivo.

The above summary of the present invention is not intended to describe each discussed embodiment of the present invention. This is the purpose of the figures and the detailed description that follows.

### BRIEF DESCRIPTION OF THE FIGURES

The invention may be more completely understood in connection with the following drawings, in which:

FIG. 1 is a scanning electron microscope image of MD-hexanoate/PEVA/PBMA/PEI-DNA coating taken at 500× magnification.

FIG. 2 is a graph showing the elution of nucleic acid delivery complexes from coated coils into phosphate buffered saline.

FIG. 3 is a fluorescence microscopy image of HEK293 cells transfected with GFP-DNA/PEI polyplexes eluted from a MD-hexanoate/PEVA/PBMA/PEI/DNA coating.

FIG. 4 is a graph showing the elution of nucleic acid delivery complexes from degradable polymer filaments.

FIG. 5 is a graph showing luciferase expression HEK-293 cells.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

As used herein, the term “complex” shall refer to a chemical association of two or more chemical species through non-covalent bonds.

As used herein, the term “dispersed” shall refer to the state of being substantially non-aggregated. By way of example, dispersed complexes shall refer to complexes that are not substantially aggregated to one another.

As used herein, the term “matrix” shall refer to a three-dimensional polymer network. In some embodiments, matrices can be disposed on a substrate, such as in the form of a coating. In other embodiments, a substrate may be omitted.

One approach to maintaining the activity of nucleic acid-based therapeutic agents is to complex the nucleic acids with a delivery agent prior to administration to a mammalian subject. By way of example, attempts have been made to complex a carrier agent, such as polyethylenimine, with nucleic acids in order to prevent degradation during the delivery phase and enhance cell entry. While such an approach can aid in preserving the activity of the nucleic acid during the delivery phase, it does not address the issue of controlled release of the nucleic acid.

One approach to providing controlled release of an active agent, in accordance with embodiments herein, is to incorporate the same in a polymeric matrix. Polymeric matrices can serve to release active agents in a controlled matter. However, the use of nucleic acid/carrier agent complexes (“nucleic acid delivery complexes”) in conjunction with polymeric matrices can raise additional issues. One issue that arises is the tendency for the resulting complexes to aggregate during processing steps associated with forming the matrix. This can be problematic because if the complexes aggregate to any significant extent, they may be unsuitable for uptake by the target cell. Another issue is that some solvents used to carry polymers that form the matrix can have adverse effects on the nucleic acid delivery complexes. Yet another issue is achieving controlled predictable release of nucleic acid delivery complexes from a polymeric matrix.

Embodiments of the present invention can include devices, matrices, and related methods that can enable delivery of nucleic acids in a site specific manner with controlled release. As shown in the examples below, the nucleic acid delivery complexes can be released from the elution control matrices in a controlled manner while retaining sufficient activity so as to be capable of transfecting target cells.

In various embodiments, the medical device can be configured to elute nucleic acid delivery complexes over an extended period of time. By way of example, in some embodiments the medical device can be configured to elute nucleic acid delivery particles for a period of time greater than or equal to two days. In some embodiments the medical device can be configured to elute nucleic acid delivery particles for a period of time greater than or equal to one week. In some embodiments the medical device can be configured to elute nucleic acid delivery particles for a period of time greater than or equal to two weeks. In some embodiments the medical device can be configured to elute nucleic acid delivery particles for a period of time greater than or equal to one month.

In various embodiments, the elution rate of the nucleic acid delivery complexes from a polymeric matrix can be controlled or tuned. By way of example, the rate of polyplex elution can be modulated by varying the number of cross-linkable groups on the polymer or by varying the concentration of the polymer in the polymer solution used to make the polymeric matrix. Embodiments of the invention also include methods of making elution control matrices with nucleic acid delivery complexes. Various aspects of exemplary embodiments will now be described in greater detail.

### Nucleic Acid Delivery Complexes

Embodiments of the invention can include elution control coatings that include nucleic acid delivery complexes. Nucleic acid delivery complexes can include a nucleic acid as an active agent and a carrier agent complexed to the nucleic acid. In some circumstances, complexes of nucleic acids and carrier agents have also been referred to as “nucleic acid delivery particles”. For purposes of this application, the terms “nucleic acid delivery complex” and “nucleic acid delivery particle” shall be interchangeable.

Carrier agents used with embodiments of the invention can include those compounds that can be complexed with nucleic acids in order to preserve the activity of the nucleic acids during the manufacturing and delivery processes. Exemplary carrier agents can also include those effective to promote internalization of nucleic acids into cells. Exemplary classes of suitable carrier agents can include cationic compounds (compounds having a net positive charge) and charge neutral compounds. By way of example, suitable carrier agents can include cationic macromolecules such as cationic polymers. Suitable carrier agents can also include cationic lipids. Suitable cationic carrier agents can also include polycation containing cyclodextrin, histones, cationized human serum albumin, aminopolysaccharides such as chitosan, peptides such as poly-L-lysine, poly-L-ornithine, and poly-4-hydroxy-L-proline ester, peptides including protein transduction domains, and polyamines such as polyethylenimine (PEI), polypropylenimine, polyamidoamine dendrimers, and poly(beta-aminoesters). Other carrier agents can include solid nucleic acid lipid nanoparticles (SNALPs), liposomes, protein transduction domains and polyvinyl pyrrolidone (PVP). Additionally, carriers may also be conjugated to molecules which allow them to target specific cell types. Examples of targeting agents include antibodies and peptides which recognize and bind to specific cell surface molecules.

Nucleic acids used with embodiments of the invention can include various types of nucleic acids that can function to provide a therapeutic effect. Exemplary types of nucleic acids can include, but are not limited to, ribonucleic acids (RNA), deoxyribonucleic acids (DNA), small interfering RNA (siRNA), micro RNA (miRNA), piwi-interacting RNA (piRNA), short hairpin RNA (shRNA), antisense nucleic acids, aptamers, ribozymes, locked nucleic acids and catalytic DNA.

Nucleic acid delivery complexes can be formed from carrier agents and nucleic acids through various processes. In some cases, for example, a cationic carrier agent interacts with an anionic nucleic acid molecule and condenses into a compact, ordered complex. As such, in some embodiments, the nucleic acid can simply be contacted with the cationic carrier agent in order to form nucleic acid delivery complexes.

The effective size of the nucleic acid delivery complexes can be affected by various factors including the molecular weight of the carrier agent, conditions during complex formation, and the molecular weight of the nucleic acid, amongst others. However, while not intending to be bound by theory, it can be desirable to control the size of the nucleic acid delivery complexes. For example, complexes that are excessively large may be much less effective at transfection than smaller complexes. In some embodiments, these complexes can have a diameter of less than about 1 micron. In some embodiments, these complexes can have a diameter of less than about 500 nm. In some embodiments, these complexes can have a diameter of less than about 200 nm. In some embodiments, these complexes can have a diameter of between about 20 and 200 nm.

Methods of Forming Coatings with Nucleic Acid Delivery Complexes

In many embodiments, the nucleic acid delivery complexes are suspended in an aqueous solvent prior to their incorporation in an elution control matrix. However, in some embodiments, the nucleic acid delivery complexes can be suspended in an organic solvent. In still other embodiments, the nucleic acid delivery complexes can be suspended in a mixed organic/aqueous solvent. However, while not intending to be bound by theory, it is believed that the use of some organic solvents to suspend the nucleic acid delivery complexes can contribute to degradation of the complexes, damaging DNA, and/or aggregation of the complexes. In some embodiments, the nucleic acid delivery complexes are suspended in a polar solvent.

Elution control matrices of embodiments herein can be made of one or more polymers, such as hydrophobic polymers, hydrophilic polymers, degradable polymers, and non-degradable polymers. Exemplary polymers are described in greater detail below. In many cases, application of these polymers to form an elution control matrix involves the use of a solvent as a carrier during the deposition process. Some of these polymers can be soluble in aqueous solvents whereas other polymers may only be soluble in organic solvents. Some of these polymers can be soluble in polar solvents whereas other polymers may only be soluble in nonpolar solvents. Solvents can specifically include water, alcohols (e.g., methanol, butanol, propanol, and isopropanol), alkanes (e.g., halogenated such as chloroform or unhalogenated alkanes such as hexane and cyclohexane), amides (e.g., dimethylformamide), ethers (e.g., THF and dioxolane), ketones (e.g., methylethylketone), aromatic compounds (e.g., toluene and xylene), nitriles (e.g., acetonitrile), esters (e.g., ethyl acetate), and combinations thereof.

In some embodiments, such as where the polymers for the elution control matrix have similar solvent requirements as the nucleic acid delivery complexes, the nucleic acid delivery complexes can simply be mixed with the polymers of the elution control matrix to form a single solution and then applied onto a substrate. However, where the polymer(s) of the elution control matrix have solvent requirements that are incompatible with the nucleic acid delivery complexes, the nucleic acid delivery complexes can be kept separate from the polymers of the elution control matrix prior to application onto a substrate. For example, one solution containing the nucleic acid delivery complexes can be formed and separately a solution containing the polymers of the elution control matrix can formed. Specifically, a nucleic acid delivery complex solution can be formed with a polar solvent and an elution control matrix polymer solution can be formed with a non-polar solvent. Then both solutions can be applied to a substrate.

It will be appreciated that many different techniques may be used to apply the polymers of the elution control coating and the nucleic acid delivery complexes onto a substrate. By way of example, techniques for applying such coatings can include spray deposition, dip coating, brush coating, printing, and the like.

However, while not intending to be bound by theory, it is believed that spray deposition can offer various advantages in the context of medical devices. For example, spray deposition, in contrast to dip coating, can allow a greater degree of precision in terms of the total amount of the active agent being deposited onto the substrate.

In addition, the spray coating process can be performed while minimizing the opportunities for phase separation that might otherwise occur when using immiscible components. For example, where the polymer(s) to be used in the elution control layer has solvent requirements that are incompatible with the nucleic acid delivery complexes, spray coating can be used to achieve a coating layer with substantially uniform dispersion of the component parts. This is because during the spray coating process the solvent(s) used can quickly evaporate without allowing a relatively large amount of solvent to build-up. Thus, since solvent is quickly removed, the remaining components have less of an opportunity to phase separate on a macroscopic scale.

In an embodiment, the invention includes a method of applying a coating including spraying a solution containing nucleic acid delivery complexes from a first spray head or nozzle while simultaneously spraying a polymer solution from a second spray head or nozzle. Spraying solutions from multiple spray heads or nozzles can offer the advantage of keeping components with incompatible solvent requirements separate until application onto the substrate. Also, it is believed that spraying solutions from multiple spray heads or nozzles can generate a more uniform dispersal of the nucleic acid delivery complexes within the elution control matrix. In some embodiments, the nucleic acid delivery complexes are substantially dispersed within the elution control matrix.

U.S. Publ. Pat. Application No. 2007/0128343, entitled “Apparatus and Methods for Applying Coatings”, the contents of which is herein incorporated by reference in its entirety, describes various techniques of applying a coating from multiple spray heads and related equipment. The spray heads or nozzles can be of a gas-atomization type or of an ultrasonic atomization type. In some embodiments, the spray heads or nozzles are ultrasonic.

It has been observed that spray coating of nucleic acid delivery complexes is desirably performed using a solution that has a relatively high concentration of the complexes. In the context of spray coating, if the concentration of the complexes in the solution is too low, applying a therapeutically significant amount of complexes to a substrate involves spraying a relatively large amount of solvent onto the substrate. The application of such a large amount of solvent can compromise the quality of the resulting elution control coating.

In addition, it can also be desirable to use a solution with a relatively high concentration of the complexes in order to form cross-linked matrices having an active agent loading high enough to achieve desirable therapeutic outcomes. In some embodiments, the solution containing the nucleic acid delivery complexes has a nucleic acid concentration of at least about 1 mg/ml.

Unfortunately, formation of nucleic acid delivery complexes, such as by simply contacting nucleic acids with cationic polymers, generally results in a solution of complexes that is not sufficiently concentrated for some uses such as spray deposition. Further, contacting nucleic acids with cationic polymers at higher concentrations in an attempt to create a complex solution with a higher concentration generally results in aggregation of the components instead of complex formation. As such, forming a complex solution that is sufficiently concentrated poses a significant challenge.

However, it has been discovered that the complexes can be formed at a lower concentration and then the resulting solution can be concentrated after complex formation in order to provide a complex solution with a desirable concentration. By way of example, a delivery complex solution can be concentrated using a centrifugal separation technique, a vacuum based concentration technique, or though other techniques. In some embodiments, the concentration of the delivery complex solution is increased to at least about 1 mg/ml of nucleic acids after complex formation. In some embodiments, the concentration of the delivery complex solution is increased to at least about 5 mg/ml of nucleic acids after complex formation. In some embodiments, the concentration of the delivery complex solution is increased to at least about 10 mg/ml of nucleic acids after complex formation.

In some embodiments, a polymeric matrix can be formed through cross-linking of a polymer. By way of example, in some embodiments, a polymer solution containing nucleic acid delivery complexes can be deposited onto a substrate and then cross-linking can be performed in situ in order to form a matrix to control elution of the nucleic acid delivery complexes. Cross-linking can be triggered in various ways. In some embodiments, a cross-linking agent or initiator can be included with the polymer solution. Different types of cross-linking agents can be used including positively charged, negatively charged, and charge neutral agents. However, while not intending to be bound by theory, it is believed that negatively charged cross-linking agents may contribute to the dissociation of nucleic acid delivery complexes. As such, in some embodiments, the cross-linking agent is positively charged or charge neutral. An example of a suitable positively charged photoinitiated crosslinking reagent is ethylenebis(4-benzoylbenzyldimethylammonium) dibromide, which can be prepared as described in example 2 of U.S. Pat. No. 5,714,360.

In some embodiments, a cross-linking agent can be activated by actinic radiation such as the application of UV light. However, UV light having relatively short wavelengths can undesirably damage nucleic acids. In some embodiments, UV light used is filtered to exclude wavelengths damaging to nucleic acids. In some embodiments, the actinic radiation comprises UV light with a wavelength of greater than or equal to 300 nm. In some embodiments, the actinic radiation comprises UV light with a wavelength of greater than or equal to 323 nm. In some embodiments, the actinic radiation comprises UV light with a wavelength of greater than or equal to 360 nm.

As such in some embodiments, the cross-linking agent is selected so that UV light having relatively long wavelengths can be effective for activation. Specifically, in some embodiments the cross-linking agent can be selected to function with light having a wavelength of equal to or greater than about 360 nm. A specific example of such an initiator is Bis(2,4,6-trimethylbenzoyl)-phenylphosphineoxide sold under the trade name IRGACURE 819 (commercially available from Ciba Specialty Chemicals, Tarrytown, N.Y.).

### Hydrophobic and Hydrophilic Polymers

As described above, embodiments can include an elution control coating that includes a polymeric matrix. In an embodiment, the polymeric matrix can include one or more hydrophobic polymers. In an embodiment, the polymeric matrix can include one or more hydrophilic polymers. One method of defining the hydrophobicity of a polymer is by the solubility parameter (or Hildebrand parameter) of the polymer. The solubility parameter describes the attractive strength between molecules of the material. The solubility parameter is represented by Equation 1:

δ=(ΔE^{v}/V)^{1/2} (Equation 1)

where δ=solubility parameter ((cal/cm³)^{1/2})

ΔE^{v}=energy of vaporization (cal)

V=molar volume (cm³)

Solubility parameters cannot be calculated for polymers from heat of vaporization data because of their nonvolatility. Accordingly, solubility parameters must be calculated indirectly. One method involves identifying solvents in which a polymer dissolves without a change in heat or volume and then defining the solubility parameter of the polymer to be the same as the solubility parameters of the identified solvents. A more complete discussion of solubility parameters and methods of calculating the same can be found in Brandup et al., Polymer Handbook, 4th Ed., John Wiley & Sons, N.Y. (1999) beginning at Chap. VII p. 675.

As a general rule, the value of the solubility parameter δ is inversely proportional to the degree of hydrophobicity of a polymer. Thus, polymers that are very hydrophobic may have a low solubility parameter value. This general proposition is particularly applicable for polymers having a glass transition temperature below physiological temperature. In an embodiment, hydrophobic polymers used with the invention have a solubility parameter less than about 11.0 (cal/cm³)^{1/2}. In an embodiment, hydrophobic polymers used with the invention have a solubility parameter of less than about 10.0 (cal/cm³)^{1/2}. In an embodiment, hydrophilic polymers used with the invention have a solubility parameter greater than about 13.0 (cal/cm³)^{1/2}. In an embodiment, hydrophilic polymers used with the invention have a solubility parameter of greater than about 14.0 (cal/cm³)^{1/2}.

### Degradable Polymers

As described above, embodiments can include an elution control coating that includes a polymeric matrix. In an embodiment, the polymeric matrix can include one or more degradable polymers. Degradable polymers used with embodiments of the invention can include both natural or synthetic polymers. Examples of degradable polymers can include those with hydrolytically unstable linkages in the polymeric backbone. Degradable polymers of the invention can include both those with bulk erosion characteristics and those with surface erosion characteristics.

While not intending to be bound by theory, the use of degradable polyesters can be advantageous in the context of providing controlled release of nucleic acid delivery complexes because release can be mediated by degradation of the matrix in addition to diffusion through the matrix.

Synthetic degradable polymers can include: degradable polyesters (such as poly(glycolic acid), poly(lactic acid), poly(lactic-co-glycolic acid), poly(dioxanone), polylactones (e.g., poly(caprolactone)), poly(3-hydroxybutyrate), poly(3-hydroxyvalerate), poly(valerolactone), poly(tartronic acid), poly(β-malonic acid), poly(propylene fumarate)); degradable polyesteramides; degradable polyanhydrides (such as poly(sebacic acid), poly(1,6-bis(carboxyphenoxy)hexane, poly(1,3-bis(carboxyphenoxy)propane); degradable polycarbonates (such as tyrosine-based polycarbonates); degradable polyiminocarbonates; degradable polyarylates (such as tyrosine-based polyarylates); degradable polyorthoesters; degradable polyurethanes; degradable polyphosphazenes; and copolymers thereof.

Natural or naturally-based degradable polymers can include polysaccharides and modified polysaccharides such as starch, cellulose, chitin, chitosan, and copolymers thereof.

Specific examples of degradable polymers include poly(ether ester) multiblock copolymers based on poly(ethylene glycol) (PEG) and poly(butylene terephthalate) that can be described by the following general structure:

[—(OCH₂CH₂)ₙ—O—C(O)—C₆H₄—C(O)-]x[-O—(CH₂)₄—O—C(O)—C₆H₄—C(O)—]y,

where —C₆H₄— designates the divalent aromatic ring residue from each esterified molecule of terephthalic acid, n represents the number of ethylene oxide units in each hydrophilic PEG block, x represents the number of hydrophilic blocks in the copolymer, and y represents the number of hydrophobic blocks in the copolymer. The subscript “n” can be selected such that the molecular weight of the PEG block is between about 300 and about 4000. The repeating units “x” and “y” can be selected so that the multiblock copolymer contains from about 55% up to about 80% PEG by weight. The block copolymer can be engineered to provide a wide array of physical characteristics (e.g., hydrophilicity, adherence, strength, malleability, degradability, durability, flexibility) and active agent release characteristics (e.g., through controlled polymer degradation and swelling) by varying the values of n, x and y in the copolymer structure. Such degradable polymers can specifically include those described in U.S. Pat. No. 5,980,948, the content of which is herein incorporated by reference in its entirety.

Degradable polyesteramides can include those formed from the monomers OH-x-OH, z, and COOH-y-COOH, wherein x is alkyl, y is alkyl, and z is leucine or phenylalanine. Such degradable polyesteramides can specifically include those described in U.S. Pat. No. 6,703,040, the content of which is herein incorporated by reference in its entirety.

Degradable polymeric materials can also be selected from: (a) non-peptide polyamino polymers; (b) polyiminocarbonates; (c) amino acid-derived polycarbonates and polyarylates; and (d) poly(alkylene oxide) polymers.

In an embodiment, the degradable polymeric material is composed of a non-peptide polyamino acid polymer. Exemplary non-peptide polyamino acid polymers are described, for example, in U.S. Pat. No. 4,638,045 (“Non-Peptide Polyamino Acid Bioerodible Polymers,” Jan. 20, 1987). Generally speaking, these polymeric materials are derived from monomers, including two or three amino acid units having one of the following two structures illustrated below:

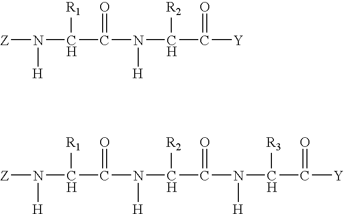

wherein the monomer units are joined via hydrolytically labile bonds at not less than one of the side groups R₁, R₂, and R₃, and where R₁, R₂, R₃ are the side chains of naturally occurring amino acids; Z is any desirable amine protecting group or hydrogen; and Y is any desirable carboxyl protecting group or hydroxyl. Each monomer unit comprises naturally occurring amino acids that are then polymerized as monomer units via linkages other than by the amide or “peptide” bond. The monomer units can be composed of two or three amino acids united through a peptide bond and thus comprise dipeptides or tripeptides. Regardless of the precise composition of the monomer unit, all are polymerized by hydrolytically labile bonds via their respective side chains rather than via the amino and carboxyl groups forming the amide bond typical of polypeptide chains. Such polymer compositions are nontoxic, are degradable, and can provide zero-order release kinetics for the delivery of active agents in a variety of therapeutic applications. According to these aspects, the amino acids are selected from naturally occurring L-alpha amino acids, including alanine, valine, leucine, isoleucine, proline, serine, threonine, aspartic acid, glutamic acid, asparagine, glutamine, lysine, hydroxylysine, arginine, hydroxyproline, methionine, cysteine, cystine, phenylalanine, tyrosine, tryptophan, histidine, citrulline, ornithine, lanthionine, hypoglycin A, β-alanine, γ-amino butyric acid, α aminoadipic acid, canavanine, venkolic acid, thiolhistidine, ergothionine, dihydroxyphenylalanine, and other amino acids well recognized and characterized in protein chemistry.

Degradable polymers of the invention can also include polymerized polysaccharides such as those described in U.S. Publ. Pat. Application No. 2005/0255142, entitled “COATINGS FOR MEDICAL ARTICLES INCLUDING NATURAL BIODEGRADABLE POLYSACCHARIDES”, U.S. Publ. Pat. Application No. 2007/0065481, entitled “COATINGS INCLUDING NATURAL BIODEGRADABLE POLYSACCHARIDES AND USES THEREOF”, and in U.S. Publ. Pat. Application No. 20070218102, entitled “HYDROPHOBIC DERIVATIVES OF NATURAL BIODEGRADABLE POLYSACCHARIDES”, all of which are herein incorporated by reference in their entirety.

Degradable polymers of the invention can also include dextran based polymers such as those described in U.S. Pat. No. 6,303,148, entitled “PROCESS FOR THE PREPARATION OF A CONTROLLED RELEASE SYSTEM”, the content of which is herein incorporated by reference in its entirety. Exemplary dextran based degradable polymers including those available commercially under the trade name OCTODEX.

Degradable polymers of the invention can further include collagen/hyaluronic acid polymers.

Degradable polymers of the invention can include multi-block copolymers, comprising at least two hydrolysable segments derived from pre-polymers A and B, which segments are linked by a multi-functional chain-extender and are chosen from the pre-polymers A and B, and triblock copolymers ABA and BAB, wherein the multi-block copolymer is amorphous and has one or more glass transition temperatures (Tg) of at most 37° C. (Tg) at physiological (body) conditions. The pre-polymers A and B can be a hydrolysable polyester, polyetherester, polycarbonate, polyestercarbonate, polyanhydride or copolymers thereof, derived from cyclic monomers such as lactide (L,D or L/D), glycolide, ε-caprolactone, δ-valerolactone, trimethylene carbonate, tetramethylene carbonate, 1,5-dioxepane-2-one, 1,4-dioxane-2-one (para-dioxanone) or cyclic anhydrides (oxepane-2,7-dione). The composition of the pre-polymers may be chosen in such a way that the maximum glass transition temperature of the resulting copolymer is below 37° C. at body conditions. To fulfill the requirement of a Tg below 37° C., some of the above-mentioned monomers or combinations of monomers may be more preferred than others. This may by itself lower the Tg, or the pre-polymer is modified with a polyethylene glycol with sufficient molecular weight to lower the glass transition temperature of the copolymer. The degradable multi-block copolymers can include hydrolysable sequences being amorphous and the segments may be linked by a multifunctional chain-extender, the segments having different physical and degradation characteristics. For example, a multi-block co-polyester consisting of a glycolide-ε-caprolactone segment and a lactide-glycolide segment can be composed of two different polyester pre-polymers. By controlling the segment monomer composition, segment ratio and length, a variety of polymers with properties that can easily be tuned can be obtained. Such degradable multi-block copolymers can specifically include those described in U.S. Publ. App. No. 2007/0155906, the content of which is herein incorporated by reference in its entirety.

### Non-Degradable Polymers

As described above, some embodiments can include a coating that includes a polymeric matrix. In an embodiment, the polymeric matrix can include a non-degradable polymer. In an embodiment, the non-degradable polymer includes a plurality of polymers, including a first polymer and a second polymer. When the coating solution contains only one polymer, it can be either a first or second polymer as described herein. As used herein, the term “(meth)acrylate”, when used in describing polymers, shall mean the form including the methyl group (methacrylate) or the form without the methyl group (acrylate).

First polymers of the invention can include a polymer selected from the group consisting of poly(alkyl(meth)acrylates) and poly(aromatic(meth)acrylates), where “(meth)” will be understood by those skilled in the art to include such molecules in either the acrylic and/or methacrylic form (corresponding to the acrylates and/or methacrylates, respectively). An exemplary first polymer is poly(n-butyl methacrylate) (pBMA). Such polymers are available commercially, e.g., from Aldrich, with molecular weights ranging from about 200,000 Daltons to about 320,000 Daltons, and with varying inherent viscosity, solubility, and form (e.g., as crystals or powder). In some embodiments, poly(n-butyl methacrylate) (pBMA) is used with a molecular weight of about 200,000 Daltons to about 300,000 Daltons.

Examples of suitable first polymers also include polymers selected from the group consisting of poly(aryl(meth)acrylates), poly(aralkyl(meth)acrylates), and poly(aryloxyalkyl(meth)acrylates). Such terms are used to describe polymeric structures wherein at least one carbon chain and at least one aromatic ring are combined with acrylic groups, typically esters, to provide a composition. In particular, exemplary polymeric structures include those with aryl groups having from 6 to 16 carbon atoms and with weight average molecular weights from about 50 to about 900 kilodaltons. Suitable poly(aralkyl(meth)acrylates), poly(arylalky(meth)acrylates) or poly(aryloxyalkyl (meth)acrylates) can be made from aromatic esters derived from alcohols also containing aromatic moieties. Examples of poly(aryl(meth)acrylates) include poly(9-anthracenyl methacrylate), poly(chlorophenylacrylate), poly(methacryloxy-2-hydroxybenzophenone), poly(methacryloxybenzotriazole), poly(naphthylacrylate) and -methacrylate), poly(4-nitrophenyl acrylate), poly(pentachloro(bromo, fluoro) acrylate) and -methacrylate), and poly(phenyl acrylate) and -methacrylate). Examples of poly(aralkyl(meth)acrylates) include poly(benzyl acrylate) and -methacrylate), poly(2-phenethyl acrylate) and -methacrylate, and poly(1-pyrenylmethyl methacrylate). Examples of poly(aryloxyalkyl (meth)acrylates) include poly(phenoxyethyl acrylate) and -methacrylate), and poly(polyethylene glycol phenyl ether acrylates) and -methacrylates with varying polyethylene glycol molecular weights.

Examples of suitable second polymers are available commercially and include poly(ethylene-co-vinyl acetate) (pEVA) having vinyl acetate concentrations of between about 10% and about 50% (12%, 14%, 18%, 25%, 33% versions are commercially available), in the form of beads, pellets, granules, etc. The pEVA co-polymers with lower percent vinyl acetate become increasingly insoluble in typical solvents, whereas those with higher percent vinyl acetate become decreasingly durable.

An exemplary polymer mixture includes mixtures of pBMA and pEVA. This mixture of polymers can be used with absolute polymer concentrations (i.e., the total combined concentrations of both polymers in the coating material), of between about 0.25 wt. % and about 99 wt. %. This mixture can also be used with individual polymer concentrations in the coating solution of between about 0.05 wt. % and about 99 wt. %. In one embodiment the polymer mixture includes pBMA with a molecular weight of from 100 kilodaltons to 900 kilodaltons and a pEVA copolymer with a vinyl acetate content of from 24 to 36 weight percent. In an embodiment the polymer mixture includes pBMA with a molecular weight of from 200 kilodaltons to 300 kilodaltons and a pEVA copolymer with a vinyl acetate content of from 24 to 36 weight percent. The concentration of the active agent or agents dissolved or suspended in the coating mixture can range from 0.01 to 99 percent, by weight, based on the weight of the final coating material.

Second polymers can also comprise one or more polymers selected from the group consisting of (i) poly(alkylene-co-alkyl(meth)acrylates, (ii) ethylene copolymers with other alkylenes, (iii) polybutenes, (iv) diolefin derived non-aromatic polymers and copolymers, (v) aromatic group-containing copolymers, and (vi) epichlorohydrin-containing polymers.

Poly(alkylene-co-alkyl(meth)acrylates) include those copolymers in which the alkyl groups are either linear or branched, and substituted or unsubstituted with non-interfering groups or atoms. Such alkyl groups can comprise from 1 to 8 carbon atoms, inclusive. Such alkyl groups can comprise from 1 to 4 carbon atoms, inclusive. In an embodiment, the alkyl group is methyl. In some embodiments, copolymers that include such alkyl groups can comprise from about 15% to about 80% (wt) of alkyl acrylate. When the alkyl group is methyl, the polymer contains from about 20% to about 40% methyl acrylate in some embodiments, and from about 25% to about 30% methyl acrylate in a particular embodiment. When the alkyl group is ethyl, the polymer contains from about 15% to about 40% ethyl acrylate in an embodiment, and when the alkyl group is butyl, the polymer contains from about 20% to about 40% butyl acrylate in an embodiment.

Alternatively, second polymers can comprise ethylene copolymers with other alkylenes, which in turn, can include straight and branched alkylenes, as well as substituted or unsubstituted alkylenes. Examples include copolymers prepared from alkylenes that comprise from 3 to 8 branched or linear carbon atoms, inclusive. In an embodiment, copolymers prepared from alkylene groups that comprise from 3 to 4 branched or linear carbon atoms, inclusive. In a particular embodiment, copolymers prepared from alkylene groups containing 3 carbon atoms (e.g., propene). By way of example, the other alkylene is a straight chain alkylene (e.g., 1-alkylene). Exemplary copolymers of this type can comprise from about 20% to about 90% (based on moles) of ethylene. In an embodiment, copolymers of this type comprise from about 35% to about 80% (mole) of ethylene. Such copolymers will have a molecular weight of between about 30 kilodaltons to about 500 kilodaltons. Exemplary copolymers are selected from the group consisting of poly(ethylene-co-propylene), poly(ethylene-co-1-butene), poly(ethylene-co-1-butene-co-1-hexene) and/or poly(ethylene-co-1-octene).

“Polybutenes” include polymers derived by homopolymerizing or randomly interpolymerizing isobutylene, 1-butene and/or 2-butene. The polybutene can be a homopolymer of any of the isomers or it can be a copolymer or a terpolymer of any of the monomers in any ratio. In an embodiment, the polybutene contains at least about 90% (wt) of isobutylene or 1-butene. In a particular embodiment, the polybutene contains at least about 90% (wt) of isobutylene. The polybutene may contain non-interfering amounts of other ingredients or additives, for instance it can contain up to 1000 ppm of an antioxidant (e.g., 2,6-di-tert-butyl-methylphenol). By way of example, the polybutene can have a molecular weight between about 150 kilodaltons and about 1,000 kilodaltons. In an embodiment, the polybutene can have between about 200 kilodaltons and about 600 kilodaltons. In a particular embodiment, the polybutene can have between about 350 kilodaltons and about 500 kilodaltons. Polybutenes having a molecular weight greater than about 600 kilodaltons, including greater than 1,000 kilodaltons are available but are expected to be more difficult to work with.

Additional alternative second polymers include diolefin-derived, non-aromatic polymers and copolymers, including those in which the diolefin monomer used to prepare the polymer or copolymer is selected from butadiene (CH₂═CH—CH═CH₂) and/or isoprene (CH₂═CH—C(CH₃)═CH₂). In an embodiment, the polymer is a homopolymer derived from diolefin monomers or is a copolymer of diolefin monomer with non-aromatic mono-olefin monomer, and optionally, the homopolymer or copolymer can be partially hydrogenated. Such polymers can be selected from the group consisting of polybutadienes prepared by the polymerization of cis-, trans- and/or 1,2-monomer units, or from a mixture of all three monomers, and polyisoprenes prepared by the polymerization of cis-1,4- and/or trans-1,4-monomer units. Alternatively, the polymer is a copolymer, including graft copolymers, and random copolymers based on a non-aromatic mono-olefin monomer such as acrylonitrile, and an alkyl (meth)acrylate and/or isobutylene. In an embodiment, when the mono-olefin monomer is acrylonitrile, the interpolymerized acrylonitrile is present at up to about 50% by weight; and when the mono-olefin monomer is isobutylene, the diolefin is isoprene (e.g., to form what is commercially known as a “butyl rubber”). Exemplary polymers and copolymers have a molecular weight between about 150 kilodaltons and about 1,000 kilodaltons. In an embodiment, polymers and copolymers have a molecular weight between about 200 kilodaltons and about 600 kilodaltons.

Additional alternative second polymers include aromatic group-containing copolymers, including random copolymers, block copolymers and graft copolymers. In an embodiment, the aromatic group is incorporated into the copolymer via the polymerization of styrene. In a particular embodiment, the random copolymer is a copolymer derived from copolymerization of styrene monomer and one or more monomers selected from butadiene, isoprene, acrylonitrile, a C₁-C₄ alkyl (meth)acrylate (e.g., methyl methacrylate) and/or butene. Useful block copolymers include copolymer containing (a) blocks of polystyrene, (b) blocks of a polyolefin selected from polybutadiene, polyisoprene and/or polybutene (e.g., isobutylene), and (c) optionally a third monomer (e.g., ethylene) copolymerized in the polyolefin block. The aromatic group-containing copolymers contain about 10% to about 50% (wt.) of polymerized aromatic monomer and the molecular weight of the copolymer is from about 300 kilodaltons to about 500 kilodaltons. In an embodiment, the molecular weight of the copolymer is from about 100 kilodaltons to about 300 kilodaltons.

Additional alternative second polymers include epichlorohydrin homopolymers and poly(epichlorohydrin-co-alkylene oxide) copolymers. In an embodiment, in the case of the copolymer, the copolymerized alkylene oxide is ethylene oxide. By way of example, epichlorohydrin content of the epichlorohydrin-containing polymer is from about 30% to 100% (wt). In an embodiment, epichlorohydrin content is from about 50% to 100% (wt). In an embodiment, the epichlorohydrin-containing polymers have a molecular weight from about 100 kilodaltons to about 300 kilodaltons.

Non-degradable polymers can also include those described in U.S. Publ. Pat. App. No. 2007/0026037, entitled “DEVICES, ARTICLES, COATINGS, AND METHODS FOR CONTROLLED ACTIVE AGENT RELEASE OR HEMOCOMPATIBILITY”, the contents of which are herein incorporated by reference in its entirety. As a specific example, non-degradable polymers can include random copolymers of butyl methacrylate-co-acrylamido-methyl-propane sulfonate (BMA-AMPS). In some embodiments, the random copolymer can include AMPS in an amount equal to about 0.5 mol. % to about 40 mol. %.

### Substrates

It will be appreciated that embodiments of the invention can be used in conjunction with various types of substrates. Exemplary substrates can include metals, polymers, ceramics, and natural materials. Metals can include, but are not limited to, cobalt, chromium, nickel, titanium, tantalum, iridium, tungsten and alloys such as stainless steel, nitinol or cobalt chromium. Suitable metals can also include the noble metals such as gold, silver, copper, platinum, and alloys including the same.

Substrate polymers include those formed of synthetic polymers, including oligomers, homopolymers, and copolymers resulting from either addition or condensation polymerizations. Examples include, but not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, styrene, vinyl chloride, vinyl acetate, vinyl pyrrolidone, and vinylidene difluoride, condensation polymers including, but are not limited to, polyamides such as polycaprolactam, polylauryl lactam, polyhexamethylene adipamide, and polyhexamethylene dodecanediamide, and also polyurethanes, polycarbonates, polysulfones, poly(ethylene terephthalate), polytetrafluoroethylene, polyethylene, polypropylene, polylactic acid, polyglycolic acid, polysiloxanes (silicones), cellulose, and polyetheretherketone.

Embodiments of the invention can also include the use of ceramics as a substrate. Ceramics include, but are not limited to, silicon nitride, silicon carbide, zirconia, and alumina, as well as glass, silica, and sapphire.

Certain natural materials can also be used in some embodiments including human tissue, when used as a component of a device, such as bone, cartilage, skin and enamel; and other organic materials such as wood, cellulose, compressed carbon, rubber, silk, wool, and cotton. Substrates can also include carbon fiber. Substrates can also include resins, polysaccharides, silicon, or silica-based materials, glass, films, gels, and membranes.

### Medical Devices

Embodiments of the invention can include and can be used with implantable, or transitorily implantable, devices including, but not limited to, vascular devices such as grafts (e.g., abdominal aortic aneurysm grafts, etc.), stents (e.g., self-expanding stents typically made from nitinol, balloon-expanded stents typically prepared from stainless steel, degradable coronary stents, etc.), catheters (including arterial, intravenous, blood pressure, stent graft, etc.), valves (e.g., polymeric or carbon mechanical valves, tissue valves, valve designs including percutaneous, sewing cuff, and the like), embolic protection filters (including distal protection devices), vena cava filters, aneurysm exclusion devices, artificial hearts, cardiac jackets, and heart assist devices (including left ventricle assist devices), implantable defibrillators, electro-stimulation devices and leads (including pacemakers, lead adapters and lead connectors), implanted medical device power supplies (e.g., batteries, etc.), peripheral cardiovascular devices, atrial septal defect closures, left atrial appendage filters, valve annuloplasty devices (e.g., annuloplasty rings), mitral valve repair devices, vascular intervention devices, ventricular assist pumps, and vascular access devices (including parenteral feeding catheters, vascular access ports, central venous access catheters); surgical devices such as sutures of all types, staples, anastomosis devices (including anastomotic closures), suture anchors, hemostatic barriers, screws, plates, clips, vascular implants, tissue scaffolds, cerebro-spinal fluid shunts, shunts for hydrocephalus, drainage tubes, catheters including thoracic cavity suction drainage catheters, abscess drainage catheters, biliary drainage products, and implantable pumps; orthopedic devices such as joint implants, acetabular cups, patellar buttons, bone repair/augmentation devices, spinal devices (e.g., vertebral disks and the like), bone pins, cartilage repair devices, and artificial tendons; dental devices such as dental implants and dental fracture repair devices; drug delivery devices such as drug delivery pumps, implanted drug infusion tubes, drug infusion catheters, and intravitreal drug delivery devices; ophthalmic devices including orbital implants, glaucoma drain shunts and intraocular lenses; urological devices such as penile devices (e.g., impotence implants), sphincter, urethral, prostate, and bladder devices (e.g., incontinence devices, benign prostate hyperplasia management devices, prostate cancer implants, etc.), urinary catheters including indwelling (“Foley”) and non-indwelling urinary catheters, and renal devices; synthetic prostheses such as breast prostheses and artificial organs (e.g., pancreas, liver, lungs, heart, etc.); respiratory devices including lung catheters; neurological devices such as neurostimulators, neurological catheters, neurovascular balloon catheters, neuro-aneurysm treatment coils, and neuropatches; ear nose and throat devices such as nasal buttons, nasal and airway splints, nasal tampons, ear wicks, ear drainage tubes, tympanostomy vent tubes, otological strips, laryngectomy tubes, esophageal tubes, esophageal stents, laryngeal stents, salivary bypass tubes, and tracheostomy tubes; biosensor devices including glucose sensors, cardiac sensors, intra-arterial blood gas sensors; oncological implants; and pain management implants.

In some aspects, embodiments of the invention can include and be utilized in conjunction with ophthalmic devices. Suitable ophthalmic devices in accordance with these aspects can provide bioactive agent to any desired area of the eye. In some aspects, the devices can be utilized to deliver bioactive agent to an anterior segment of the eye (in front of the lens), and/or a posterior segment of the eye (behind the lens). Suitable ophthalmic devices can also be utilized to provide bioactive agent to tissues in proximity to the eye, when desired.

In some aspects, embodiments of the invention can be utilized in conjunction with ophthalmic devices configured for placement at an external or internal site of the eye. Suitable external devices can be configured for topical administration of bioactive agent. Such external devices can reside on an external surface of the eye, such as the cornea (for example, contact lenses) or bulbar conjunctiva. In some embodiments, suitable external devices can reside in proximity to an external surface of the eye.

Devices configured for placement at an internal site of the eye can reside within any desired area of the eye. In some aspects, the ophthalmic devices can be configured for placement at an intraocular site, such as the vitreous. Illustrative intraocular devices include, but are not limited to, those described in U.S. Pat. No. 6,719,750 B2 (“Devices for Intraocular Drug Delivery,” Varner et al.) and U.S. Pat. No. 5,466,233 (“Tack for Intraocular Drug Delivery and Method for Inserting and Removing Same,” Weiner et al.); U.S. Publication Nos. 2005/0019371 A1 (“Controlled Release Bioactive Agent Delivery Device,” Anderson et al.), 2004/0133155 A1 (“Devices for Intraocular Drug Delivery,” Varner et al.), 2005/0059956 A1 (“Devices for Intraocular Drug Delivery,” Varner et al.), and 2003/0014036 A1 (“Reservoir Device for Intraocular Drug Delivery,” Varner et al.); and U.S. application Ser. Nos. 11/204,195 (filed Aug. 15, 2005, Anderson et al.), 11/204,271 (filed Aug. 15, 2005, Anderson et al.), 11/203,981 (filed Aug. 15, 2005, Anderson et al.), 11/203,879 (filed Aug. 15, 2005, Anderson et al.), 11/203,931 (filed Aug. 15, 2005, Anderson et al.); and related applications.

In some aspects, the ophthalmic devices can be configured for placement at a subretinal area within the eye. Illustrative ophthalmic devices for subretinal application include, but are not limited to, those described in U.S. Patent Publication No. 2005/0143363 (“Method for Subretinal Administration of Therapeutics Including Steroids; Method for Localizing Pharmacodynamic Action at the Choroid and the Retina; and Related Methods for Treatment and/or Prevention of Retinal Diseases,” de Juan et al.); U.S. application Ser. No. 11/175,850 (“Methods and Devices for the Treatment of Ocular Conditions,” de Juan et al.); and related applications.

Suitable ophthalmic devices can be configured for placement within any desired tissues of the eye. For example, ophthalmic devices can be configured for placement at a subconjunctival area of the eye, such as devices positioned extrasclerally but under the conjunctiva, such as glaucoma drainage devices and the like.

It will be appreciated that embodiments of the invention can also be used without substrates. By way of example, embodiments can include a matrix with nucleic acid delivery complexes disposed therein in the form of a filament or other shape without including a substrate.

The present invention may be better understood with reference to the following examples. These examples are intended to be representative of specific embodiments of the invention, and are not intended as limiting the scope of the invention.

### EXAMPLES

### Example 1

### Preparation of MD-Hexanoate Macromer

A 4 g portion of dried Maltodextrin (MO40, obtained from Grain Processing Corporation, Muscatine, Iowa) was dissolved in 40 mls of dimethyl sulfoxide with stirring. When the solution was complete, 9.48 g (0.12 moles, 9.16 mls) of 1-methylimidizole followed by 24.63 g (0.12 moles, 26.6 mls) of caproic anhydride were added with stirring at room temperature. The reaction solution was stirred for one hour and was then slowly add to 750 mls of deionized water in a Waring blender. The precipitated solid was collected via filtration, re-suspended in 1 L of deionized water and stirred for one hour. The solid obtained was taffy-like and collected via filtration and dried in vacuo. 7.18 g of a white solid was obtained. The theoretical degree of substitution (DS) was 2.5.

### Example 2

### Preparation of Maltodextrin-Methacrylate Macromer (MD-Methacrylate)

Maltodextrin (100 g; 33.3 mmole; Dextrose Equivalent: 4.0-7.0; Sigma-Aldrich, Milwaukee, Wis.) was dissolved in dimethylsulfoxide (DMSO) 1,000 mL with stirring. The size of the maltodextrin was calculated to be in the range of 2,000-4,000 Daltons. Once the reaction solution was complete, either 1.86 g (22.65 mmole) of 1-methylimidazole (Sigma-Aldrich, Milwaukee, Wis.) followed by 3.49 g (22.65 mmole) of methacrylic-anhydride (Sigma-Aldrich, Milwaukee, Wis.) or 1.16 g (14.18 mmole) of 1-methylimidazole followed by 2.19 g (14.18 mmole) methacrylic-anhydride were added to separate portions of the reaction solution with stirring. The MD-methacrylate produced by these two procedures had a theoretical Degree of Substitution (DS) of either 0.4 or 0.2, respectively. The reaction mixtures were further stirred for one hour at room temperature. After this time, the reaction mixtures were quenched with water and dialyzed against DI water using 1,000 MWCO dialysis tubing. The MD-methacrylate was isolated via lyophilization. As determined by NMR, the actual load of methacrylate on the two MD-methacrylate macromers was 0.402 (DS=0.4) and 0.213 (DS=0.2) μmoles/mg.

### Example 3

### Nucleic Acid Delivery Complex Formation

DNA-PEI nucleic acid delivery complexes were fabricated based on published methods (Boussif, O., et al. “A versatile vector for gene and oligonucleotide transfer into cells in culture and in vivo: polyethylenimine.” Proceedings Natiopnal Academy of Sciences. 92.16 (August 1995): 7297-7301.). In brief, 1 mL of aqueous DNA was added to an equal volume of PEI (branched polyethyleneimine 25 kDa obtained from Sigma-Aldrich, St. Louis, Mo.) in aqueous solution and the mixture was vortexed at room temperature for 30 seconds to produce a nitrogen to phosphate (N/P) ratio of 15 (calculated based on the number of primary amines in PEI and the number of phosphate groups in DNA). Multiple batches of nucleic acid delivery complexes were combined and total DNA content was measured by UV-VIS at 260 nm.

### Example 4

### Concentration of Nucleic Acid Delivery Complex Solution

An aqueous solution of nucleic acid delivery complexes was prepared as described in example 3 above. The solution was estimated to have a concentration of roughly 0.05 mg DNA/ml. The solution was then concentrated to approximately 1.6 mg/ml of DNA by sequential centrifugal filtration using a Microsep filter (Pall Corp.) with a 10K Mw cut-off. The concentrated solution was observed by visual inspection to be substantially free of nucleic acid delivery complex aggregation.

### Example 5

### Formation of Coatings with Nucleic Acid Delivery Complexes

A first polymer solution was formed by combining “PA” (poly(butyleneterephthalate-co-ethylene glycol) copolymer with 80 wt. % polyethylene glycol (1000PEG80PBT20) having an average molecular weight of 1000 kD and 20 wt. % butyleneterephthalate) obtained from OctoPlus, Cambridge, Mass.) with “PEVA” (poly(ethylene-co-vinyl acetate) 33 wt % vinyl acetate, obtained from Sigma-Aldrich, St. Louis, Mo.) and “PBMA” (poly(n-butyl methacrylate) 337 kD, obtained from Sigma-Aldrich, St. Louis, Mo.) in a solvent of chloroform to achieve a total solids concentration of 3.2 mg/ml (37.5% PA, 31.25% PEVA, and 31.25% PBMA).

A second polymer solution was formed by combining MD-hexanoate (as formed in example 1) with PEVA and PBMA in a solvent of chloroform to achieve a total solids concentration of 3.2 mg/ml (37.5% MD-hexanoate polymer, 31.25% PEVA, and 31.25% PBMA).

Nucleic acid delivery complex containing coatings were then deposited onto metal coils as described in US Publ. Pat. Appl. 2005/0019371. A spraying apparatus, as described in U.S. Publ. Pat. App. No. 2007/0128343, was used to simultaneously deposit a concentrated nucleic acid delivery complex solution formed as described in example 4 above and either the first (n=4 coils) or the second (n=4 coils) polymer solution in an environment of roughly 3 percent relative humidity. Specifically, the concentrated nucleic acid delivery complex solution was applied from a first ultrasonic spray head at a rate of approximately 0.015 mls/min while either the first or the second polymer solution was applied from a second ultrasonic spray head at a rate of approximately 0.03 mls/min. The first set of coils (1, 2, 3, and 4) had a solids composition of 20 wt. % DNA/PEI, 30 wt. % PA, 25 wt. % PEVA, and 25 wt. % PBMA. The second set of coils (5, 6, 7, and 8) had a solids composition of 20 wt. % DNA/PEI, 30 wt. % MD-hexanoate, 25 wt. % PEVA, and 25 wt. % PBMA. The amounts of total solids (including DNA/PEI) and DNA alone are summarized below in Table 1.

TABLE 1
Total coating weight and DNA content for coated coils.

| | Coil | Total Solids | DNA |
|---|---|---|---|
| PA/PEVA/PBMA/PEI/DNA | 1 | 153 μg | 31 μg |
| | 2 | 156 μg | 31 μg |
| | 3 | 143 μg | 29 μg |
| | 4 | 150 μg | 30 μg |
| MD-hexanoate/PEVA/PBMA/PEI/DNA | 5 | 163 μg | 33 μg |
| | 6 | 172 μg | 34 μg |
| | 7 | 158 μg | 32 μg |
| | 8 | 168 μg | 34 μg |

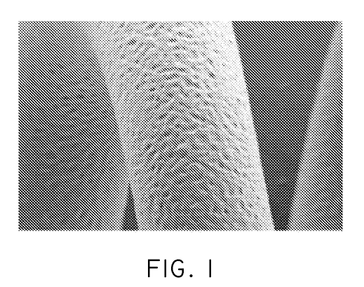

The coated coils were then dried under ambient conditions. Visual inspection revealed a smooth conformal coating. The coated coils were examined with scanning electron microscopy (SEM) and Raman spectroscopy. SEM analysis revealed that the coils had a uniform surface that appeared to have a roughness characterized by 3 to 5 μm size features in a dimple pattern. FIG. 1 shows a scanning electron microscope image of MD-hexanoate /PEVA/PBMA/PEI-DNA coating taken at 500× magnification.

Raman spectroscopy revealed that the nucleic acid delivery complexes were dispersed within the coating. In general the PEI and DNA were concentrated in the lower dimple areas, while MD-hexanoate/PEVA/PBMA were concentrated in the higher, raised regions of the coating. Similarly, Raman spectroscopy showed that PA/PEVA/PBMA were concentrated in the higher, raised regions of the coating.

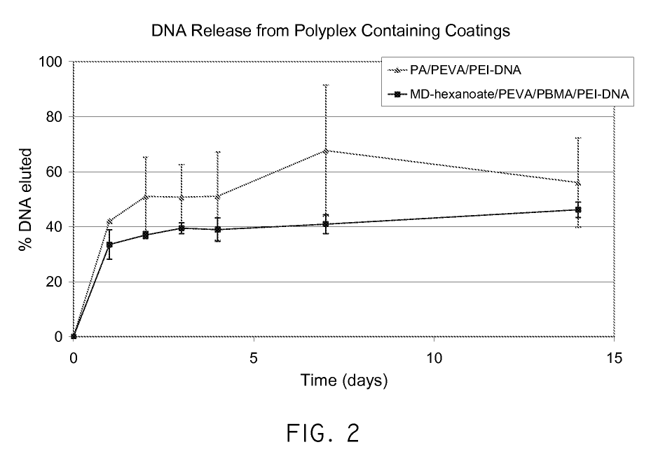

Elution of the nucleic acid delivery complexes from the coated coils was then assessed. Specifically, the coils were deposited in 200 μl of sterile, RNase/DNase free water at 37 degrees Celsius. Eluent was sampled every 24 hours for the first four days and on days 7 and 14. DNA concentration was determined by UV-VIS absorption at 260 nm. The results of the elution testing are shown below in Table 2 and in FIG. 2.

TABLE 2
Summary of % DNA eluted from coating in vitro.

Ave % DNA Eluted

| | MD-hexanoate/PEVA/ | PA/PEVA/ |
|---|---|---|
| Time (days) | PBMA/PEI/DNA | PBMA/PEI/DNA |
| 0 | 0 | 0 |
| 1 | 33.5 ± 5.4 | 41.0 ± 0.4 |
| 2 | 37.0 ± 1.3 | 51.0 ± 14.2 |
| 3 | 39.4 ± 2.0 | 51.0 ± 11.9 |
| 4 | 38.9 ± 4.2 | 51.0 ± 16.1 |
| 7 | 40.9 ± 3.4 | 67.7 ± 23.8 |
| 14 | 46.1 ± 2.7 | 56.0 ± 16.2 |

### Example 6

### Activity of Nucleic Acid Delivery Complexes Released from Coils

A nucleic acid delivery complex containing coating was applied to four coils according to the procedure described in example 5 above. The coating included a solids content of 20 wt. % DNA/PEI, 30 wt. % MD-hexanoate, 25 wt. % PEVA, and 25 wt. % PBMA.

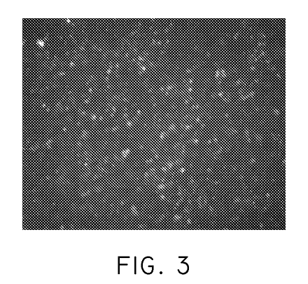

The nucleic acid delivery complexes were then eluted into an elution media. Specifically, the coils were separately placed into 200 ul of EMEM (serum free media from Gibco/Invitrogen, Carlsbad, Calif.) under sterile conditions and incubated at 37 degrees Celsius for 48 hours. The eluent was then withdrawn and used to transfect HEK293 human epithelial kidney cells (obtained from American Type Culture Collection (ATCC, Manassas, Va.). Specifically, cells were transfected for 24 hours in the presence of 10% FBS serum, and transfection efficiency was evaluated after 3 days in culture by imaging cells under fluorescent microscopy to observe GFP (green fluorescent protein) production in transfected cells (see FIG. 3, for example). Fluorescence was observed in the transfected cells indicating that activity of the nucleic acid delivery complexes was retained through the coating and elution processes described above.

### Example 7

### Effect of Concentration and Crosslink Density of Methacrylate Modified Starch Controls Elution of DNA/PEI Complexes

DNA was labeled with Cy3 according to the instructions of the manufacturer in the labeling kit (Cy3 DNA labeling kit, Mirus Bio, Madison, Wis.). PEI/DNA complexes were formed in H₂O with 100 mg/ml DNA at an N/P ratio of 5. MD-Methacrylate (DS=0.2 or 0.4, from example 2 above) was dissolved in 100 μl of polyplex solution at concentrations of 500 or 1000 mg/ml. Irgacure (Irgacure 819DW, Ciba Specialty Chemicals, Tarrytown, N.Y.) dissolved in methanol at 50 mg/ml was diluted 1:10 into the MD-Methacrylate solution and the resulting solution was placed in silicone tubing with an inner diameter of 0.0625 inches. Solutions were then exposed to a UV light source (Bluewave 200, Dymax, Torrington, Conn.) with a 324 nm high pass filter for 2 minutes to form crosslinked filaments containing 10 μg of DNA. Filaments were cut into 4 equal pieces and were placed in 0.5 ml of sterile phosphate buffered saline (PBS). PBS was replaced at specified time points and Cy3 levels in removed PBS were determined using a fluorescent plate reader with appropriate filters. The amount of eluted polyplex was determined by comparing to a standard curve of PEI/DNA complexes generated by serial dilution in PBS.

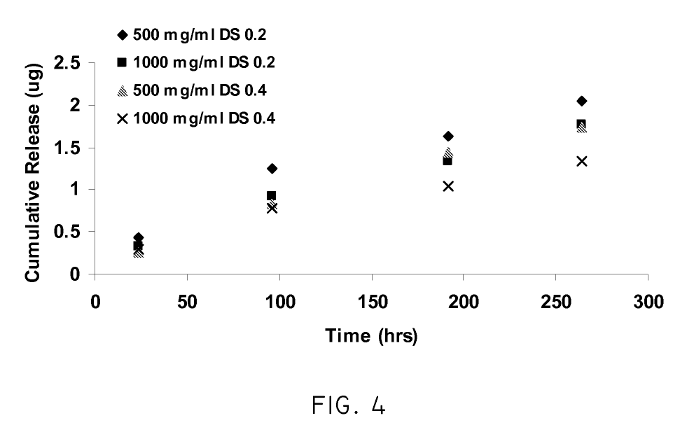

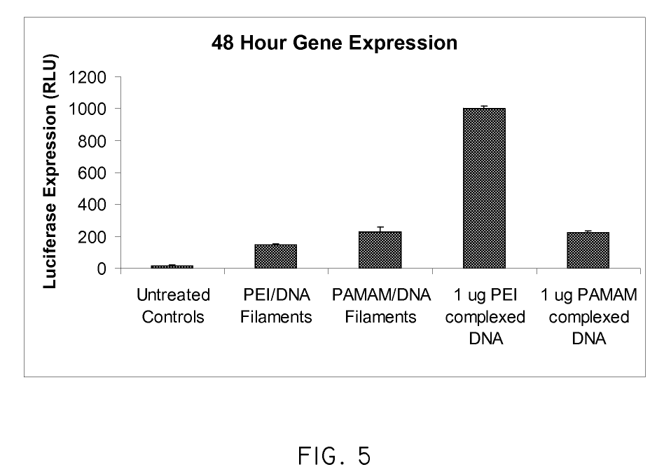

As demonstrated in FIG. 4, the rate of polyplex elution can be controlled by either the degree of methacrylate modification (e.g., varying the number of cross-linkable groups on the polymer) or the concentration of maltodextrin (e.g. varying the concentration of the polymer in the polymer solution). The fastest elution of polyplexes was seen from filaments prepared at 500 mg/ml using maltodextrin with a methacrylate degree of substitution of 0.2 (diamonds in FIG. 5). At 264 hours approximately 80% of the initial loading of 2.5 μg/filament of polyplex was released from these filaments. The slowest rate of polyplex release was seen with filaments prepared at 1000 mg/ml using maltodextrin with a methacrylate degree of substitution of 0.4 (x in FIG. 5), while intermediate release rates were seen with high concentrations of maltodextrin at low DS (squares) or low concentrations with high DS (triangles).

### Example 8

### Complexes of Cationic Polymers and DNA Released from Crosslinked Filaments of Methacrylate Modified Starch Transfect Cells

PEI/DNA and polyamidoamine (PAMAM) dendrimer/DNA complexes were formed in H₂O at a 10 μg/ml concentration of DNA. Specifically, 300 μg of DNA (gWiz Luciferase, Aldevron, Fargo, N. Dak.) was dissolved in 15 ml of H₂O. PEI or PAMAM dendrimer (Sigma, St. Louis, Mo., Generation 5, ethylenediamine core) were dissolved in H₂O at 900 μg/ml or 1050 μg/ml respectively. Polymer solutions were combined with DNA, vortexed and incubated 10 minutes to form complexes.

Solutions were then concentrated 100 times to 1 mg/ml DNA by centrifugation under a vacuum. MD-Methacrylate (DS=0.4, from example 2 above) was dissolved in H₂O at 1000 mg/ml. Ethylenebis(4-benzoylbenzyldimethylammonium) dibromide, prepared as described in example 2 of U.S. Pat. No. 5,714,360, was dissolved at 5 mg/ml in H₂O. 60 μl of complexes were combined with 60 μl of MD-Methacrylate and 12 μl of ethylenebis(4-benzoylbenzyldimethylammonium) dibromide solution and transferred to silicone tubing with an inner diameter of 0.0625 inches.

Solutions were then exposed to a UV light source (Bluewave 200, Dymax, Torrington, Conn.) with a 324 nm high pass filter for 1 minute to form crosslinked filaments containing 60 μg of DNA. These filaments were then sectioned into 6 pieces each containing approximately 10 μg of complexed DNA each. Sections were placed in 24 well plates containing 1×10⁵ HEK-293 cells in 500 μl of media containing 10% FBS. As controls, 1 ug of PEI and PAMAM DNA complexes were added to cells or cells were left untreated. Filaments were incubated with cells for 48 hours. Filaments and media were then removed and lysed in 200 μl of cell culture lysis reagent (CCLR, Promega, Madison, Wis.). 20 μl of cell lysates were combined in a white 96 well plate with 100 μl of Promega Luciferase Assay Reagent and read to determine luciferase expression in cells. The data are shown in FIG. 5.

As shown in FIG. 5, luciferase expression was observed in cells exposed to crosslinked filaments containing DNA complexed with both PEI and PAMAM. This example indicates that the activity of prepared complexes is retained during the crosslinking process.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference. Nothing herein is to be construed as an admission that the inventors are not entitled to antedate any publication and/or patent, including any publication and/or patent cited herein.

The invention has been described with reference to various specific and preferred embodiments and techniques. However, it should be understood that many variations and modifications may be made while remaining within the spirit and scope of the invention.

### Further Embodiments

In an embodiment the invention includes an implantable medical device including a substrate and a coating disposed on a surface of the substrate, the coating comprising a polymeric matrix and a plurality of disperse nucleic acid delivery complexes disposed within the polymeric matrix, the nucleic acid delivery complexes comprising a nucleic acid and a carrier agent complexed to the nucleic acid, the nucleic acid delivery complexes comprising an average size of less than about 1 micron, the coating configured to elute the nucleic acid delivery complexes in vivo. The implantable medical device can be configured to provide controlled elution of the nucleic acid delivery complexes. The coating can be conformal to the surface of the substrate. The polymeric matrix can include a hydrophilic polymer. The polymeric matrix can include a hydrophobic polymer. The polymeric matrix can include a degradable polymer. The polymeric matrix can include a durable polymer. The carrier agent can be effective to facilitate transfection of a host cell. The carrier agent configured to prevent degradation of the nucleic acid. The carrier agent can have a net positive charge. The carrier agent can be a cationic polymer. The carrier agent can include polyethylenimine. The carrier agent can be a cationic lipid. The nucleic acid can be selected from the group consisting of RNA, DNA, siRNA, miRNA, piRNA, shRNA, antisense nucleic acids, aptamers, ribozymes, and catalytic DNA.

In an embodiment, the invention can include an implantable medical device including a polymeric matrix and a plurality of disperse nucleic acid delivery complexes disposed within the polymeric matrix, the nucleic acid delivery complexes comprising a nucleic acid and a carrier agent; the matrix configured to elute the nucleic acid delivery complexes in vivo.

In an embodiment, the invention can include a method of making a medical device, the method including complexing nucleic acids with a carrier agent to form a delivery complex solution comprising nucleic acid delivery complexes, increasing the concentration of the delivery complex solution, applying the delivery complex solution to a substrate, and applying a polymeric solution to the substrate. Applying the polymeric solution to the substrate can be performed simultaneously with applying the delivery complex solution to the substrate. Applying the delivery complex solution to the substrate can include spraying the delivery complex solution onto the substrate. Applying the polymeric solution to the substrate can include spraying the polymeric solution onto the substrate. Increasing the concentration of the delivery complex solution can include removing a solvent while preventing aggregation of the nucleic acid delivery complexes.

In an embodiment, the invention can include a method of making a medical device, the method including complexing nucleic acids with a carrier agent to form nucleic acid delivery complexes, and combining the nucleic acid delivery complexes with a polymer solution and a cross-linking agent, wherein the cross-linking agent is positively charged or charge neutral. In an embodiment, the invention can include activating the cross-linking agent with actinic radiation. In an embodiment, the actinic radiation can include UV light with a wavelength of greater than or equal to 360 nm.

1. A method of making a medical device, the method comprising
complexing nucleic acids with a carrier agent to form a delivery complex solution comprising nucleic acid delivery complexes;
applying the delivery complex solution to a substrate; and
applying a polymeric solution to the substrate.

2. The method of claim 1, further comprising increasing the concentration of the delivery complex solution to at least about 1 mg/ml of nucleic acids after complexing the nucleic acids with the carrier agent and before applying the delivery complex solution to the substrate.

3. The method of claim 1, wherein increasing the concentration of the delivery complex solution comprises removing a solvent while preventing aggregation of the nucleic acid delivery complexes.

4. The method of claim 1, wherein applying the delivery complex solution to the substrate comprises spraying the delivery complex solution onto the substrate.

5. The method of claim 1, wherein applying the polymeric solution to the substrate comprising spraying the polymeric solution onto the substrate.

6. The method of claim 1, wherein applying the polymeric solution to the substrate is performed simultaneously with applying the delivery complex solution to the substrate.

7. The method of claim 6, wherein the polymeric solution is sprayed onto the substrate from a first spray head and the delivery complex solution is sprayed onto the substrate from a second spray head.

8. The method of claim 1, the carrier agent effective to promote internalization of nucleic acids into cells.

9. The method of claim 1, the carrier agent comprising a cationic macromolecule.

10. The method of claim 1, the carrier agent comprising a cationic polymer.

11. The method of claim 1, the carrier agent comprising polyethylenimine.

12. The method of claim 1, the carrier agent comprising a cationic lipid.

13. The method of claim 1, the carrier agent comprising a protein transduction domain.

14. The method of claim 1, the nucleic acid selected from the group consisting of RNA, DNA, miRNA, piRNA, shRNA, antisense nucleic acids, aptamers, ribozymes, and catalytic DNA.

15. The method of claim 1, the nucleic acid comprising siRNA.

16. The method of claim 1, the medical device configured to elute nucleic acid delivery complexes for a period of time greater than or equal to two weeks.

17. A method of making a medical device, the method comprising
complexing nucleic acids with a carrier agent to form nucleic acid delivery complexes;
combining the nucleic acid delivery complexes with a polymer solution and a cross-linking agent, the polymer solution comprising a polymer, wherein the cross-linking agent is positively charged or charge neutral.

18. The method of claim 17, further comprising activating the cross-linking agent with actinic radiation.

19. The method of claim 17, wherein the actinic radiation is filtered to exclude wavelengths damaging to nucleic acids.

20. The method of claim 17, the cross-linking agent comprising ethylenebis(4-benzoylbenzyldimethylammonium) dibromide.

21. The method of claim 17, the polymer solution comprising maltodextrin.

22. The method of claim 17, further comprising modulating elution rate by varying the concentration of the polymer in the polymer solution.

23. The method of claim 17, further comprising modulating elution rate by varying the number of cross-linkable groups on the polymer in the polymer solution.

24. An implantable medical device comprising:
a substrate; and
a coating disposed on a surface of the substrate, the coating comprising a polymeric matrix and a plurality of disperse nucleic acid delivery complexes disposed within the polymeric matrix,
the polymeric matrix comprising a degradable polymer and a non-degradable polymer, the nucleic acid delivery complexes comprising a nucleic acid and a carrier agent complexed to the nucleic acid, the coating configured to elute the nucleic acid delivery complexes in vivo.

25. The implantable medical device of claim 24, the degradable polymer comprising a polysaccharide containing polymer.

26. The implantable medical device of claim 24, the degradable polymer comprising maltodextrin.

27. The implantable medical device of claim 24, the non-degradable polymer comprising polyethylene-co-vinyl acetate (PEVA).

28. The implantable medical device of claim 24, the non-degradable polymer comprising a mixture of polyethylene-co-vinyl acetate (PEVA) and poly-n-butyl methacrylate (PBMA).
